(19) United States
(12) Patent Application Publication
(10) Pub. No.: US 2024/0148272 A1
(43) Pub. Date: May 9, 2024

(54) SYSTEMS, APPARATUSES, AND METHODS FOR DETECTING AIR BREATHED BY A USER

(71) Applicant: Honeywell Safety Products USA, Inc., Charlotte, NC (US)

(72) Inventors: Yan YU, Charlotte, NC (US); Zhixiong HOU, Charlotte, NC (US); Chuang HUANG, Charlotte, NC (US); Jiangtao LIU, Charlotte, NC (US); Yongfang HUANG, Charlotte, NC (US); Linan ZHAO, Charlotte, NC (US); Rui LUO, Charlotte, NC (US)

(21) Appl. No.: 18/494,304

(22) Filed: Oct. 25, 2023

(30) Foreign Application Priority Data
Nov. 9, 2022 (CN) 202211399793.X

(51) Int. Cl. A61B 5/08; A61B 5/00; A61B 5/097

(52) U.S. Cl. CPC A61B 5/082; A61B 5/0075; A61B 5/097; A61B 5/6803

(57) ABSTRACT

Systems, apparatus, and methods for detecting air breathed by a user and/or one or more gases are provided herein. In some embodiments, an apparatus may include a test material adapted to reduce in size when exposed to one or more stimuli. In some embodiments, one or more chemicals may be disposed on the test material and, when exposed to the air breathed by the user, the one or more chemicals are adapted to transform from a first color to a second color. In some embodiments, when the test material reduces in size an intensity of the second color increases. The apparatus may further include a face mask. In this regard, the test material may be removably attached to the face mask. The apparatus may further include a spectrophotometer configured to determine if the one or more chemicals have transformed from the first color to the second color.

### CROSS-REFERENCE TO RELATED APPLICATIONS

This application claims priority pursuant to 35 U.S.C. 119(a) to Chinese Application No. 202211399793.X, filed Nov. 9, 2022, which application is incorporated herein by reference in its entirety.

### TECHNOLOGICAL FIELD

Embodiments of the present disclosure relate generally to systems, apparatuses, and methods for detecting air breathed by a user and/or one or more gases.

### BACKGROUND

Applicant has identified many technical challenges and difficulties associated with systems, apparatuses, and methods for detecting air breathed by a user and/or one or more gases. Through applied effort, ingenuity, and innovation, Applicant has solved problems related to systems, apparatuses, and methods for detecting air breathed by a user and/or one or more gases by developing solutions embodied in the present disclosure, which are described in detail below.

### BRIEF SUMMARY

Various embodiments described herein relate to systems, apparatuses, and methods for detecting air breathed by a user and/or one or more gases.

In accordance with one aspect of the disclosure, an apparatus for detecting air breathed by a user is provided. In some embodiments, the apparatus includes a test material adapted to reduce in size when exposed to one or more stimuli. In some embodiments, one or more chemicals are disposed on the test material. In some embodiments, when exposed to the air breathed by the user, the one or more chemicals are adapted to transform from a first color to a second color. In some embodiments, when the test material reduces in size an intensity of the second color increases.

In some embodiments, the one or more stimuli comprise heat, light, a basic solution, an acidic solution, a gas, or moisture.

In some embodiments, the air breathed by the user comprises at least one of the one or more stimuli.

In some embodiments, the test material is adapted to reduce in size by between 1 percent and 90 percent.

In some embodiments, the apparatus further includes a face mask. In some embodiments, the test material is removably attached to the face mask.

In some embodiments, the second color is configured to provide an indication to dispose of the face mask.

In some embodiments, the one or more chemicals are further adapted to transform from the first color to the second color when exposed to one or more gases.

In some embodiments, the test material comprises one or more of a polyurethane polymer, polystyrene polymer, blended yarn, or metamaterial.

In some embodiments, the air breathed by the user comprises an indication that the user is suffering from a health problem.

In some embodiments, the apparatus further includes a spectrophotometer. In some embodiments, the spectrophotometer is configured to determine if the one or more chemicals have transformed from the first color to the second color.

In accordance with another aspect of the disclosure, a method for detecting air breathed by a user is provided. In some embodiments, the method includes allowing one or more chemicals disposed on a test material to be exposed to the air breathed by the user. In some embodiments, when exposed to the air breathed by the user, the one or more chemicals are adapted to transform from a first color to a second color. In some embodiments, the method includes exposing the test material to one or more stimuli. In some embodiments, exposing the test material to the one or more stimuli causes the test material to reduce in size. In some embodiments, the test material reducing in size causes an intensity of the second color to increase.

In some embodiments, the one or more stimuli comprise heat, light, a basic solution, an acidic solution, a gas, or moisture.

In some embodiments, the air breathed by the user comprises at least one of the one or more stimuli.

In some embodiments, the test material is adapted to reduce in size by between 1 percent and 90 percent.

In some embodiments, the test material is removably attached to a face mask.

In some embodiments, the second color is configured to provide an indication to dispose of the face mask.

In some embodiments, the one or more chemicals are further adapted to transform from the first color to the second color when exposed to one or more gases.

In some embodiments, the test material comprises one or more of a polyurethane polymer, polystyrene polymer, blended yarn, or metamaterial.

In some embodiments, the air breathed by the user comprises an indication that the user is suffering from a health problem.

In some embodiments, the method further includes determining if the one or more chemicals have transformed from the first color to the second color using a spectrophotometer.

The above summary is provided merely for purposes of summarizing some example embodiments to provide a basic understanding of some aspects of the present disclosure. Accordingly, it will be appreciated that the above-described embodiments are merely examples and should not be construed to narrow the scope or spirit of the disclosure in any way. It will be appreciated that the scope of the present disclosure encompasses many potential embodiments in addition to those here summarized, some of which will be further described below.

### BRIEF DESCRIPTION OF THE DRAWINGS

Reference will now be made to the accompanying drawings. The components illustrated in the figures may or may not be present in certain embodiments described herein. Some embodiments may include fewer (or more) components than those shown in the figures in accordance with an example embodiment of the present disclosure.

FIG. 1 illustrates a front view of an example apparatus for detecting air breathed by a user and/or one or more gases in accordance with one or more embodiments of the present disclosure;

FIG. 2 illustrates a cross sectional view of the example apparatus for detecting air breathed by a user and/or one or more gases in accordance with one or more embodiments of the present disclosure;

FIG. 3 illustrates a front view of a face mask of the example apparatus for detecting air breathed by a user and/or one or more gases in accordance with one or more embodiments of the present disclosure;

FIG. 4 illustrates a schematic view of an example spectrophotometer of the example apparatus for detecting air breathed by a user and/or one or more gases in accordance with one or more embodiments of the present disclosure;

FIG. 5 illustrates a front view of an example testing housing of the example apparatus for detecting air breathed by a user and/or one or more gases in accordance with one or more embodiments of the present disclosure;

FIG. 6 illustrates a flowchart of an example method of detecting air breathed by a user and/or one or more gases in accordance with one or more embodiments of the present disclosure; and

FIG. 7 illustrates a block diagram of an example computer processing device in accordance with one or more embodiments of the present disclosure.

### DETAILED DESCRIPTION

Example embodiments will be described more fully hereinafter with reference to the accompanying drawings, in which some, but not all embodiments of disclosure are shown. Indeed, embodiments of the disclosure may be embodied in many different forms and should not be construed as limited to the embodiments set forth herein; rather, these embodiments are provided so that this disclosure will satisfy applicable legal requirements. Like numbers refer to like elements throughout.

### Overview

Example embodiments disclosed herein address technical problems associated with systems, apparatuses, and methods for detecting air breathed by a user and/or one or more gases. As would be understood by one skilled in the field to which this disclosure pertains, there are numerous example scenarios in which detecting air breathed by a user and/or one or more gases is desirable.

In many applications, it is often necessary to detect air breathed by a user and/or one or more gases. For example, it may be necessary to detect air breathed by a user who is wearing a face mask to ensure that the face mask is functioning properly. As another example, it may be necessary to detect air breathed by a user who is undergoing a medical procedure to monitor the user's health and/or the medical procedure. As another example, it may be necessary to detect the air breathed by a user to determine if the user is suffering from a health problem. As another example, it may be necessary to detect one or more gases present in an environment to determine what type of personal protective equipment (e.g., what type of face mask) a user should wear to operate safely in the environment. As another example, it may be necessary to detect one or more gases present in a gas flow to ensure that a system associated with the gas flow is operating safely and/or properly. As another example, it may be necessary to detect one or more gases to determine the properties of the one or more gases.

Example solutions for detecting air breathed by a user and/or one or more gases include, for example, using a material that reacts when exposed to air breathed by a user and/or one or more gases to indicate that air breathed by a user and/or one or more gases has been detected. In some examples, after the material has reacted when exposed to the air breathed by a user and/or the one or more gases, the material may be inspected, either by a user visually observing the material and/or using a spectrophotometer. However, in some examples, it is often difficult to determine whether the material has reacted by inspecting the material. That is, in some examples, it is difficult for a user to visually observe any change in the material after it has reacted and/or for a spectrophotometer to determine that the material has reacted. As a result, in some examples, although the material may react when exposed to air breathed by the user and/or one or more gases, the material's practical utility is limited by the difficulty in determining whether the material has reacted by visually observing the material and/or using a spectrophotometer.

Thus, to address these and/or other issues related to detecting air breathed by a user and/or one or more gases, example systems, apparatuses, and/or methods for detecting air breathed by a user and/or one or more gases are disclosed herein. For example, an embodiment in this disclosure, described in greater detail below, includes an apparatus for detecting air breathed by a user and/or one or more gases. In some examples, the apparatus may include a test material adapted to reduce in size when exposed to one or more stimuli. In some examples, one or more chemicals may be disposed on the test material and, when exposed to the air breathed by the user and/or the one or more gases, the one or more chemicals are adapted to transform from a first color to a second color. In some examples, when the test material reduces in size when exposed to one or more stimuli an intensity of the second color increases. Accordingly, in some examples, since the test material reducing in size causes an intensity of the second color to increase, inspection of the test material (e.g., by a user visually observing the test material and/or using a spectrophotometer) can readily reveal whether air breathed by a user and/or one or more gases has been detected.

### Example Apparatus for Detecting Air Breathed by a User and/or One or More Gases

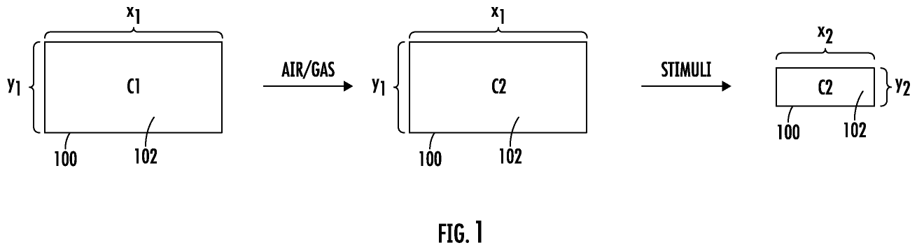

With reference to FIGS. 1-4, embodiments herein provide for an example apparatus for detecting air breathed by a user and/or one or more gases.

In some embodiments, the apparatus for detecting air breathed by a user and/or one or more gases may include a test material 100. In some embodiments, the test material 100 may be adapted to reduce in size. For example, a surface area and/or a volume of the test material 100 may be adapted to reduce in size. In this regard, for example, the test material 100 may initially have dimensions x₁, y₁, and z₁. After the test material 100 reduces in size the test material 100 may have dimensions x₂, y₂, and z₂, in which x₂ is less than x₁, y₂ is less than y₁, and/or z₂ is less than z₁, such that the surface area and/or volume of the test material 100 is less than before the test material 100 reduced in size. In some embodiments, the test material 100 may be adapted to reduce in size by between 1 percent and 90 percent. That is, the surface area and/or volume of the test material 100 may be adapted to reduce in size by between 1 percent and 90 percent. For example, the test material 100 may be adapted to reduce in size by approximately 50 percent.

In some embodiments, the test material 100 may be adapted to reduce in size when exposed to one or more stimuli. The one or more stimuli may include one or more of heat, light, a basic solution, an acidic solution, a gas, and/or moisture. For example, the test material 100 may be adapted to reduce in size when exposed to heat above a particular temperature, a specific wavelength of light, a solution above a particular pH, a solution below a particular pH, a concentration of a particular gas above a predetermined threshold (e.g., a concentration of nitric oxide in the air breathed by a user above a predetermined threshold or a concentration of nitric oxide in a flow of one or more gases above a predetermined threshold), and/or a particular amount of water vapor.

In some embodiments, one or more chemicals 102 may be disposed on the test material 100. For example, the one or more chemicals 102 may be disposed on the surface of the test material 100. The one or more chemicals 102 may be disposed over the entire surface of the test material 100. Alternatively, the one or more chemicals 102 may only be disposed on only a portion of the test material 100. The one or more chemicals 102 may include one or more organic compounds and/or non-organic compounds. For example, the one or more chemicals 102 may be one or more dye solutions.

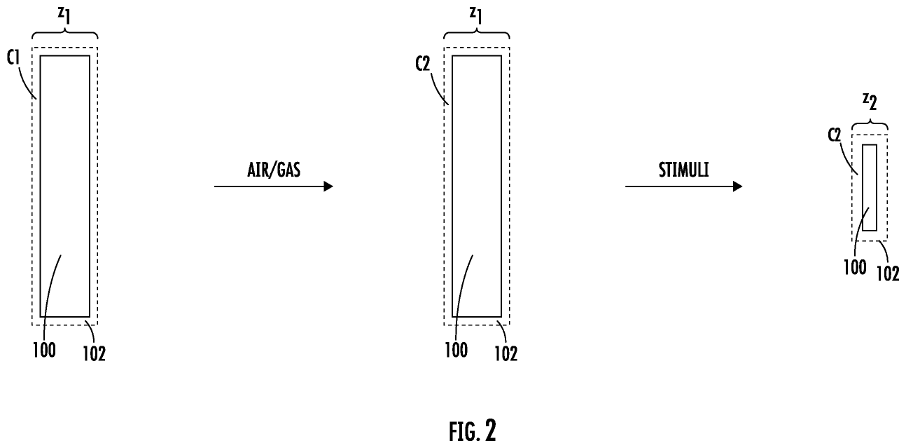

In some embodiments, when exposed to the air breathed by the user and/or the one or more gases, the one or more chemicals 102 may be adapted to transform from a first color (C1) to a second color (C2). For example, as depicted in FIGS. 1 and 2, the one or more chemicals 102 may initially be a first color (C1) and, after being exposed to air breathed by a user, the one or more chemicals 102 may transform into a second color (C2). As another example, as depicted in FIGS. 1 and 2, the one or more chemicals 102 may initially be the first color (C1) and, after being exposed to one or more gases, the one or more chemicals 102 may transform into the second color (C2). In some embodiments, the first color (C1) may be lighter than the second color (C2). For example, in some embodiments, the first color (C1) may be a light blue and the second color (C2) may be a dark blue. In other embodiments, the first color (C1) may be darker than the second color (C2). In some embodiments, the transformation of the one or more chemicals 102 from the first color (C1) to the second color (C2) may be visible to the user. That is, the one or more chemicals 102 may appear visually different to the user after the one or more chemicals 102 have transformed from the first color (C1) to the second color (C2). In some embodiments, the transformation of the one or more chemicals 102 from the first color (C1) to the second color (C2) may not be visible to the user. That is, the one or more chemicals 102 may not appear visually different to the user after the one or more chemicals 102 have transformed from the first color (C1) to the second color (C2).

In some embodiments, when the test material 100 reduces in size an intensity of the second color (C2) may increase. In this regard, when the test material 100 reduces in size, a concentration of the one or more chemicals 102 disposed on the test material 100 may increase, causing an intensity of the second color (C2) to increase. In some embodiments, the increase in intensity of the second color (C2) may make the one or more chemicals 102 visually observable to the user. Said differently, in some embodiments, the transformation of the one or more chemicals 102 from the first color (C1) to the second color (C2), may not be visually observable to the user until the test material 100 reduces in size and increases the intensity of the second color (C2).

In some embodiments, the one or more chemicals 102 may be exposed to the air breathed by the user and/or the one or more gases before the test material 100 is exposed to the one or more stimuli. As a result, for example, the one or more chemicals 102 may transform from the first color (C1) to the second color (C2) before the test material 100 reduces in size. In this regard, when the one or more chemicals 102 are exposed to the air breathed by the user and/or the one or more gases before the test material 100 is exposed to the one or more stimuli, the transformation of the one or more chemicals 102 from the first color (C1) to the second color (C2) may not be visually observable to the user because the intensity of the second color (C2) has not increased. That is, the one or more chemicals 102 may not appear visually different to the user after the one or more chemicals 102 have transformed from the first color (C1) to the second color (C2) (e.g., because the test material 100 has not reduced in size).

In some embodiments, the one or more chemicals 102 may be exposed to the air breathed by the user and/or the one or more gases after the test material 100 is exposed to the one or more stimuli. As a result, for example, the one or more chemicals 102 may transform from the first color (C1) to the second color (C2) after the test material 100 reduces in size. In this regard, when the one or more chemicals 102 are exposed to the air breathed by the user and/or the one or more gases after the test material 100 is exposed to the one or more stimuli, the transformation of the one or more chemicals 102 from the first color (C1) to the second color (C2) may be visually observable to user because the intensity of the second color (C2) has increased. That is, the one or more chemicals 102 may appear visually different to the user after the one or more chemicals 102 have transformed from the first color (C1) to the second color (C2) (e.g., because the test material 100 has reduced in size).

In some embodiments, the one or more chemicals 102 may be exposed to the air breathed by the user and/or one or more gases at substantially the same time as the test material 100 is exposed to the one or more stimuli. As a result, for example, the one or more chemicals 102 may transform from the first color (C1) to the second color (C2) at substantially the same time as the test material 100 reduces in size. In this regard, when the one or more chemicals 102 are exposed to the air breathed by the user and/or the one or more gases at substantially the same time as the test material 100 is exposed to the one or more stimuli, the transformation of the one or more chemicals 102 from the first color (C1) to the second color (C2) may be visually observable to the user because the intensity of the second color (C2) has increased. That is, the one or more chemicals 102 may appear visually different to the user after the one or more chemicals 102 have transformed from the first color (C1) to the second color (C2) (e.g., because the test material 100 has reduced in size).

In some embodiments, the one or more gases may include one of the one or more stimuli. In this regard, the one or more chemicals 102 may transform from the first color (C1) to the second color (C2) when exposed to the one or more gases and the test material 100 may reduce in size when exposed to the one or more gases (e.g., because the one or more gases includes one of the one or more stimuli). In some embodiments, the air breathed by the user may include one of the one or more stimuli. In this regard, the one or more chemicals 102 may transform from the first color (C1) to the second color (C2) when exposed to the air breathed by the user and the test material 100 may reduce in size when exposed to the air breathed by the user (e.g., because the air breathed by the user includes one of the one or more stimuli).

In some embodiments, the air breathed by the user may include an indication that the user is suffering from a health problem. In this regard, the air breathed by the user including an indication that the user is suffering from a health problem may be based on the air breathed by the user including one or more stimuli. For example, if the air breathed by the user is above a particular temperature, it may indicate that the user has a temperature. As another example, if the air breathed by the user is above or below a particular pH level, it may indicate that the user is suffering from an infection. As another example, if the air breathed by the user includes a concentration of a particular gas above a predetermined threshold, it may indicate that the user has a medical condition (e.g., if the air breathed by the user includes a concentration of nitric oxide above a particular threshold it may indicate that the user is suffering from asthma). As a result, in some embodiments, if the air breathed by the user causes the test material 100 to reduce in size (e.g., because the air breathed by the user includes one or more stimuli), the air breathed by the user includes an indication that the user is suffering from a health problem.

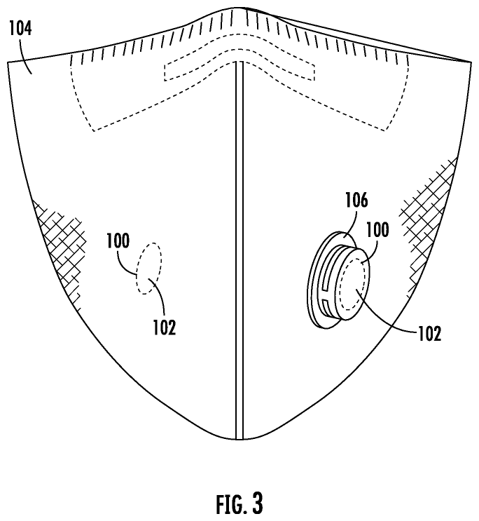

In some embodiments, such as depicted in FIG. 3, the apparatus for detecting air breathed by a user and/or the one or more gases may include a face mask 104. The face mask 104 may be configured to be worn by the user. For example, the face mask 104 may be worn by a user for which the apparatus is detecting air breathed from and/or a user is who using the apparatus to detect one or more gases. The face mask 104 may comprise woven fabric, non-woven fabric, and/or plastic. The face mask 104 may be configured to allow air breathed by the user to flow through the face mask 104 when the user is wearing the face mask 104. The face mask 104 may be configured to prevent the user from breathing at least some of one or more gases present in ambient air around the user (e.g., one or more gases outside the face mask 104) when the user is wearing the face mask 104.

In some embodiments, the face mask 104 may include a housing 106. The housing 106 may be removably attached to the face mask 104. The housing 106 may be configured to allow air breathed by the user to exit the face mask 104 while the user is wearing the face mask 104 (e.g., when the user exhales). Additionally or alternatively, the housing 106 may be configured to allow air to enter the face mask 104 (e.g., when the user inhales) while the user is wearing the face mask 104. The housing 106 may be configured to prevent the user from breathing at least some of the one or more gases present in the ambient air around the user when the user is wearing the face mask 104.

In some embodiments, the test material 100 may be disposed on or within a portion of the face mask 104. In some embodiments, such as depicted in FIG. 3, the face mask 104 may include more than one test material 100. For example, the test material 100 may comprise one of one or more layers of the face mask 104. Additionally or alternatively, the test material 100 may be disposed within the housing 106. Accordingly, the test material 100 may be exposed to air exhaled by a user wearing the face mask 104 (e.g., air exhaled by the user that flows through layers of the face mask 104 and/or air exhaled by the user that flows through the housing 106) and/or air inhaled by a user wearing the face mask 104 (e.g., air inhaled by the user that flows through layers of the face mask 104 and/or air inhaled by the user that flows through the housing 106). Similarly, the test material 100 may be exposed to one or more gases present in the ambient air around the user when the user is wearing the face mask 104. In some embodiments, the test material 100 may be removably attached to the face mask 104. For example, the test material 100 may be disposed on the face mask 104 via a hook and loop fastener. As another example, the test material 100 may be disposed within the housing 106 and the housing 106 may be removably attached to the face mask 104. In some embodiments, when the test material 100 disposed on or more within a portion of the face mask 104, the second color (C2) may provide an indication to dispose of the face mask 104.

In some embodiments, the apparatus for detecting air breathed by a user and/or one or more gases may include a spectrophotometer 410. The spectrophotometer 410 may be configured to determine if the one or more chemicals 102 have transformed from the first color (C1) to the second color (C2). For example, the test material 100 may be removed from the face mask 104 and placed inside the spectrophotometer 410 to determine if the one or more chemicals 102 have transformed from the first color (C1) to the second color (C2). As another example, the spectrophotometer 410 may be configured to determine if the one or more chemicals 102 have transformed from the first color (C1) to the second color (C2) while the test material 100 is still attached to the face mask 104. In some embodiments, the spectrophotometer 410 may be configured to determine a difference between the first color (C1) and the second color (C2). In this regard, for example, the spectrophotometer 410 may be configured to determine a difference between an intensity of the first color (C1) and an intensity of the second color (C2).

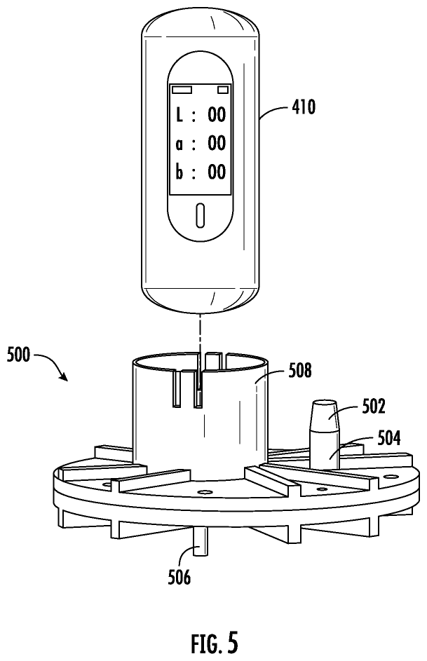

In some embodiments, such as depicted in FIG. 5, the apparatus for detecting air breathed by a user and/or one or more gases may include a testing housing 500. The testing housing 500 may be configured to facilitate detecting air breathed by the user and/or the one or more gases. In this regard, the testing housing 500 may include an inlet 502 and an outlet 506. The air breathed by the user and/or the one or more gases may enter the testing housing 500 via the inlet 502 and the air breathed by the user and/or the one or more gases may exit the testing housing 500 via the outlet 506.

In some embodiments, the testing housing 500 may further include a tube 504 in which the test material 100 is placed. The tube 504 may be in fluid communication with the inlet 502 and the outlet 506. In this regard, for example, the air breathed by the user and/or the one or more gases may enter the testing housing 500 via the inlet 502, flow through the tube 504 in which the test material 100 may be exposed to the air breathed by the user and/or the one or more gases, and the air breathed by the user and/or the one or more gases may exit the testing housing 500 via the outlet 506 after passing through the tube 504. In some embodiments, the tube 504 may be removably attached to the testing housing 500. In this regard, for example, the test material 100 may be replaced each time air breathed by the user and/or one or more gases are detected (e.g., a new test material 100 may be placed inside the tube 504). Additionally or alternatively, the tube 504 may be replaced each time air breathed by the user and/or one or more gases are detected (e.g., a new tube 504 with a new test material 100 may be attached to the testing housing 500).

In some embodiments, the testing housing 500 may further include an electronics receptacle 508. The electronics receptacle 508 may be configured to receive one or more electronic devices that may be used to facilitate detecting air exhaled by a user and/or one or more gases. For example, the electronics receptacle 508 may be configured to receive the spectrophotometer 410. In this regard, when the spectrophotometer 410 is placed in the electronics receptacle 508, the spectrophotometer 410 may be able to determine if the one or more chemicals 102 disposed on the test material 100 in the tube 504 have transformed from the first color (C1) to the second color (C2).

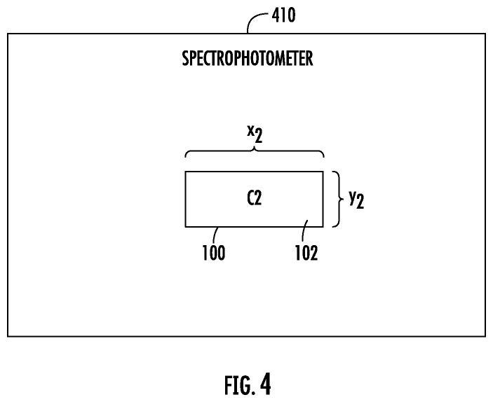

Although depicted in FIGS. 4 and 5 and described herein as the apparatus including a spectrophotometer (e.g., spectrophotometer 410) for determining if the one or more chemicals 102 have transformed from the first color (C1) to the second color (C2), it would be understood by one skilled in the field to which this disclosure pertains that one or more other devices could be used for determining if the one or more chemicals 102 have transformed from the first color (C1) to the second color (C2). For example, a colorimeter could be used for determining if the one or more chemicals 102 have transformed from the first color (C1) to the second color (C2).

In some embodiments, the test material 100 may any shape suitable for reducing in size when exposed to the one or more stimuli, for being integrated into the face mask 104, and/or for being integrated into the testing housing 500 (e.g., being placed in the tube 504). In this regard, the test material 100 may be square, rectangular, and/or circular. For example, the test material 100 may be substantially rectangular when the test material 100 is disposed within or on the face mask 104. As another example, the test material 100 may be substantially circular when the test material 100 is disposed within the housing 106 (e.g., if the housing 106 is substantially circular) and/or in the tube 504.

In some embodiments, the test material 100 may be comprised of one or more materials capable of reducing in size when exposed to the one or more stimuli. For example, the test material 100 may be one or more of a polyurethane polymer, a polystyrene polymer, a blended yarn (e.g. a yarn blended with plastics), a metamaterial (e.g., a micro-structured metamaterial having a negative thermal-expansion coefficient). In some embodiments, the test material 100 may be 3-D printed. For example, a 3-D printer may be used to create a micro-structured metamaterial having a negative thermal-expansion coefficient.

### Example Method of Detecting Air Breathed by a User and/or One or More Gases

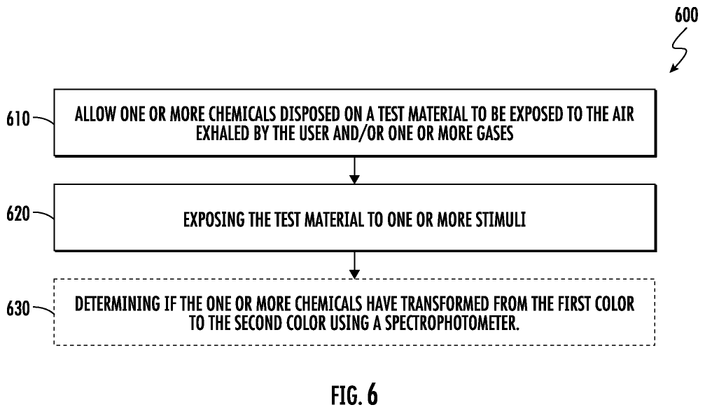

Referring now to FIG. 6, a flowchart of an example method 600 of detecting air breathed by a user and/or one or more gases is illustrated. In this regard, FIG. 6, illustrates operations that may be performed by the apparatus for detecting air breathed by a user and/or one or more gases and/or components of the apparatus for detecting air breathed by a user and/or one or more gases.

As shown in block 610, the method 600 of detecting air breathed by a user and/or one or more gases may include allowing one or more chemicals disposed on a test material to be exposed to the air breathed by the user and/or the one or more gases. As described above, the one or more chemicals may include one or more organic compounds and/or non-organic compounds. For example, the one or more chemicals may be one or more dye solutions.

As described above, when exposed to the air breathed by the user and/or the one or more gases, the one or more chemicals may be adapted to transform from a first color to a second color. For example, the one or more chemicals may initially be a first color and, after being exposed to air breathed by a user, the one or more chemicals may transform into a second color. As another example, the one or more chemicals may initially be the first color and, after being exposed to one or more gases, the one or more chemicals may transform into the second color.

As shown in block 620, the method 600 of detecting air breathed by a user and/or one or more gases may include exposing the test material to one or more stimuli. As described above, the test material may be adapted to reduce in size. For example, a surface area and/or a volume of the test material may be adapted to reduce in size. In some embodiments, the test material may be adapted to reduce in size when exposed to one or more stimuli. The one or more stimuli may include one or more of heat, light, a basic solution, an acidic solution, a gas, and/or moisture. For example, the test material may be adapted to reduce in size when exposed to heat above a particular temperature, a specific wavelength of light, a solution above a particular pH, a solution below a particular pH, a concentration of a particular gas above a predetermined threshold (e.g., a concentration of nitric oxide in the air breathed by a user above a predetermined threshold or a concentration of nitric oxide in a flow of one or more gases above a predetermined threshold), and/or a particular amount of water vapor.

As described above, in some embodiments, when the test material reduces in size an intensity of the second color may increase. In this regard, when the test material reduces in size, a concentration of the one or more chemicals disposed on the test material may increase, causing an intensity of the second color to increase. In some embodiments, the increase in intensity of the second color may make the one or more chemicals visually observable to the user. Said differently, in some embodiments, the transformation of the one or more chemicals from the first color to the second color, may not be visually observable to the user until the test material reduces in size and increases the intensity of the second color.

As described above, in some embodiments, the one or more gases may include one of the one or more stimuli. In this regard, the one or more chemicals may transform from the first color to the second color when exposed to the one or more gases and the test material may reduce in size when exposed to the one or more gases (e.g., because the one or more gases includes one of the one or more stimuli). In some embodiments, the air breathed by the user may include one of the one or more stimuli. In this regard, the one or more chemicals may transform from the first color to the second color when exposed to the air breathed by the user and the test material may reduce in size when exposed to the air breathed by the user (e.g., because the air breathed by the user includes one of the one or more stimuli).

As described above, the air breathed by the user may include an indication that the user is suffering from a health problem. In this regard, the air breathed by the user including an indication that the user is suffering from a health problem may be based on the air breathed by the user including one or more stimuli. For example, if the air breathed by the user is above a particular temperature, it may indicate that the user has a temperature. As another example, if the air breathed by the user is above or below a particular pH level, it may indicate that the user is suffering from an infection. As another example, if the air breathed by the user includes a concentration of a particular gas above a predetermined threshold, it may indicate that the user has a medical condition (e.g., if the air breathed by the user includes a concentration of nitric oxide above a particular threshold it may indicate that the user is suffering from asthma). As a result, in some embodiments, if the air breathed by the user causes the test material to reduce in size (e.g., because the air breathed by the user includes one or more stimuli), the air breathed by the user includes an indication that the user is suffering from a health problem

As described above, the test material may be disposed on or within a portion of the face mask. In some embodiments, the face mask may include more than one test material. For example, the test material may comprise one of one or more layers of the face mask. Additionally or alternatively, the test material may be disposed within the housing. Accordingly, the test material may be exposed to air exhaled by a user wearing the face mask (e.g., air exhaled by the user that flows through layers of the face mask and/or air exhaled by the user that flows through the housing) and/or air inhaled by a user wearing the face mask (e.g., air inhaled by the user that flows through layers of the face mask and/or air inhaled by the user that flows through the housing). Similarly, the test material may be exposed to one or more gases present in the ambient air around the user when the user is wearing the face mask. In some embodiments, the test material may be removably attached to the face mask.

As shown in block 630, the method 600 of detecting air breathed by a user and/or one or more gases may optionally include determining if the one or more chemicals have transformed from the first color to the second color using a spectrophotometer. As described above, spectrophotometer may be configured to determine if the one or more chemicals have transformed from the first color to the second color. For example, the test material may be removed from the face mask and placed inside the spectrophotometer to determine if the one or more chemicals have transformed from the first color to the second color. As another example, the spectrophotometer may be configured to determine if the one or more chemicals have transformed from the first color to the second color while the test material is still attached to the face mask. Although described herein as the method 600 optionally including determining if the one or more chemicals have transformed from the first color to the second color using a spectrophotometer, it would be understood by one skilled in the field to which this disclosure pertains that one or more other devices could be used for determining if the one more chemicals have transformed from the first color to the second color. For example, a colorimeter could be used for determining if the one or more chemicals have transformed from the first color to the second color.

### Example Computer Processing Device

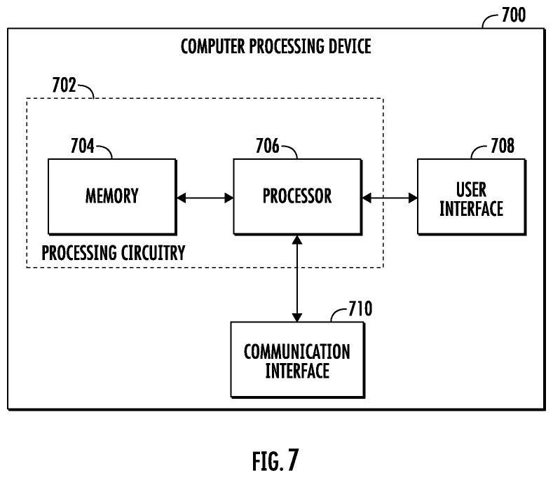

With reference to FIG. 7, a block diagram of an example computer processing device 700 is illustrated in accordance with some example embodiments. In some embodiments, the spectrophotometer 410 (or colorimeter) may include one or more computer processing devices, such as the computer processing device 700 in FIG. 7. However, it should be noted that the components, devices, or elements illustrated in and described with respect to FIG. 7 below may not be mandatory and thus one or more may be omitted in certain embodiments. Additionally, some embodiments may include further or different components, devices or elements beyond those illustrated in and described with respect to FIG. 7.

The computer processing device 700 may include or otherwise be in communication with processing circuitry 702 that is configurable to perform actions in accordance with one or more embodiments disclosed herein. In this regard, the processing circuitry 702 may be configured to perform and/or control performance of one or more functionalities of the computer processing device 700 in accordance with various embodiments, and thus may provide means for performing functionalities of the computer processing device 700 in accordance with various embodiments. The processing circuitry 702 may be configured to perform data processing, application execution and/or other processing and management services according to one or more embodiments. In some embodiments, the computer processing device 700 or a portion(s) or component(s) thereof, such as the processing circuitry 702, may be embodied as or comprise a chip or chip set. In other words, the computer processing device 700 or the processing circuitry 702 may comprise one or more physical packages (e.g., chips) including materials, components and/or wires on a structural assembly (e.g., a baseboard). The structural assembly may provide physical strength, conservation of size, and/or limitation of electrical interaction for component circuitry included thereon. The computer processing device 700 or the processing circuitry 702 may therefore, in some cases, be configured to implement an embodiment of the disclosure on a single chip or as a single “system on a chip.” As such, in some cases, a chip or chipset may constitute means for performing one or more operations for providing the functionalities described herein.

In some embodiments, the processing circuitry 702 may include a processor 706 and, in some embodiments, such as that illustrated in FIG. 5, may further include memory 704. The processing circuitry 702 may be in communication with or otherwise control a user interface 708 and/or a communication interface 710. As such, the processing circuitry 702 may be embodied as a circuit chip (e.g., an integrated circuit chip) configured (e.g., with hardware, software or a combination of hardware and software) to perform operations described herein.

The processor 706 may be embodied in a number of different ways. For example, the processor 706 may be embodied as various processing means such as one or more of a microprocessor or other processing element, a coprocessor, a controller or various other computing or processing devices including integrated circuits such as, for example, an ASIC (application specific integrated circuit), an FPGA (field programmable gate array), or the like. Although illustrated as a single processor, it will be appreciated that the processor 706 may comprise a plurality of processors. The plurality of processors may be in operative communication with each other and may be collectively configured to perform one or more functionalities of the computer processing device 700 as described herein. In some embodiments, the processor 706 may be configured to execute instructions stored in the memory 704 or otherwise accessible to the processor 706. As such, whether configured by hardware or by a combination of hardware and software, the processor 706 may represent an entity (e.g., physically embodied in circuitry—in the form of processing circuitry 702) capable of performing operations according to embodiments of the present disclosure while configured accordingly. Thus, for example, when the processor 706 is embodied as an ASIC, FPGA or the like, the processor 706 may be specifically configured hardware for conducting the operations described herein. Alternatively, as another example, when the processor 706 is embodied as an executor of software instructions, the instructions may specifically configure the processor 706 to perform one or more operations described herein.

In some embodiments, the memory 704 may include one or more non-transitory memory devices such as, for example, volatile and/or non-volatile memory that may be either fixed or removable. In this regard, the memory 704 may comprise a non-transitory computer-readable storage medium. It will be appreciated that while the memory 704 is illustrated as a single memory, the memory 704 may comprise a plurality of memories. The memory 704 may be configured to store information, data, applications, instructions and/or the like for enabling the computer processing device 700 to carry out various functions in accordance with one or more embodiments. For example, the memory 704 may be configured to buffer input data for processing by the processor 706. Additionally or alternatively, the memory 704 may be configured to store instructions for execution by the processor 706. As yet another alternative, the memory 704 may include one or more databases that may store a variety of files, contents or data sets. Among the contents of the memory 704, applications may be stored for execution by the processor 706 in order to carry out the functionality associated with each respective application. In some cases, the memory 704 may be in communication with one or more of the processor 706, user interface 708, and/or communication interface 710 via a bus(es) for passing information among components of the computer processing device 700.

The user interface 708 may be in communication with the processing circuitry 702 to receive an indication of a user input at the user interface 708 and/or to provide an audible, visual, mechanical or other output to the user. As such, the user interface 708 may include, for example, a keyboard, a mouse, a joystick, a display, a touch screen display, a microphone, a speaker, and/or other input/output mechanisms. As such, the user interface 708 may, in some embodiments, provide means for a user to access and interact with the spectrophotometer 410 (or colorimeter).

The communication interface 710 may include one or more interface mechanisms for enabling communication with other devices and/or networks. In some cases, the communication interface 710 may be any means such as a device or circuitry embodied in either hardware, or a combination of hardware and software that is configured to receive and/or transmit data from/to a network and/or any other device or module in communication with the processing circuitry 702. By way of example, the communication interface 710 may be configured to enable the spectrophotometer 410 (or colorimeter) to communicate with other computer processing devices. Accordingly, the communication interface 710 may, for example, include an antenna (or multiple antennas) and supporting hardware and/or software for enabling communications with a wireless communication network (e.g., a wireless local area network, cellular network, global positing system network, and/or the like) and/or a communication modem or other hardware/software for supporting communication via cable, digital subscriber line (DSL), universal serial bus (USB), Ethernet or other methods.

Many modifications and other embodiments of the inventions set forth herein will come to mind to one skilled in the art to which these inventions pertain having the benefit of teachings presented in the foregoing descriptions and the associated drawings. Although the figures only show certain components of the apparatus and systems described herein, it is understood that various other components may be used in conjunction with the system. Therefore, it is to be understood that the inventions are not to be limited to the specific embodiments disclosed and that modifications and other embodiments are intended to be included within the scope of the appended claims. Moreover, the steps in the method described above may not necessarily occur in the order depicted in the accompanying diagrams, and in some cases one or more of the steps depicted may occur substantially simultaneously, or additional steps may be involved. Although specific terms are employed herein, they are used in a generic and descriptive sense only and not for purposes of limitation.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure.

Use of broader terms such as “comprises,” “includes,” and “having” should be understood to provide support for narrower terms such as “consisting of,” “consisting essentially of,” and “comprised substantially of” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

That which is claimed:

1. An apparatus for detecting air breathed by a user, the apparatus comprising:
a test material adapted to reduce in size when exposed to one or more stimuli, wherein:
one or more chemicals are disposed on the test material,
when exposed to the air breathed by the user, the one or more chemicals are adapted to transform from a first color to a second color, and
when the test material reduces in size an intensity of the second color increases.

2. The apparatus of claim 1, wherein the one or more stimuli comprise heat, light, a basic solution, an acidic solution, a gas, or moisture.

3. The apparatus of claim 1, wherein the air breathed by the user comprises at least one of the one or more stimuli.

4. The apparatus of claim 1, wherein the test material is adapted to reduce in size by between 1 percent and 90 percent.

5. The apparatus of claim 1, further comprising:
a face mask, wherein the test material is removably attached to the face mask.

6. The apparatus of claim 5, wherein the second color is configured to provide an indication to dispose of the face mask.

7. The apparatus of claim 1, wherein the one or more chemicals are further adapted to transform from the first color to the second color when exposed to one or more gases.

8. The apparatus of claim 1, wherein the test material comprises one or more of a polyurethane polymer, polystyrene polymer, blended yarn, or metamaterial.

9. The apparatus of claim 1, wherein the air breathed by the user comprises an indication that the user is suffering from a health problem.

10. The apparatus of claim 1, further comprising:
a spectrophotometer, wherein the spectrophotometer is configured to determine if the one or more chemicals have transformed from the first color to the second color.

11. A method of detecting air breathed by a user, the method comprising:
allowing one or more chemicals disposed on a test material to be exposed to the air breathed by the user, wherein, when exposed to the air breathed by the user, the one or more chemicals are adapted to transform from a first color to a second color; and
exposing the test material to one or more stimuli, wherein exposing the test material to the one or more stimuli causes the test material to reduce in size, wherein the test material reducing in size causes an intensity of the second color to increase.

12. The method of claim 11, wherein the one or more stimuli comprise heat, light, a basic solution, an acidic solution, a gas, or moisture.

13. The method of claim 11, wherein the air breathed by the user comprises at least one of the one or more stimuli.

14. The method of claim 11, wherein the test material is adapted to reduce in size by between 1 percent and 90 percent.

15. The method of claim 11, wherein the test material is removably attached to a face mask.

16. The method of claim 15, wherein the second color is configured to provide an indication to dispose of the face mask.

17. The method of claim 11, wherein the one or more chemicals are further adapted to transform from the first color to the second color when exposed to one or more gases.

18. The method of claim 11, wherein the test material comprises one or more of a polyurethane polymer, polystyrene polymer, blended yarn, or metamaterial.

19. The method of claim 11, wherein the air breathed by the user comprises an indication that the user is suffering from a health problem.

20. The method of claim 11, further comprising:
determining if the one or more chemicals have transformed from the first color to the second color using a spectrophotometer.
